(19) United States
(12) Patent Application Publication
(10) Pub. No.: US 2022/0315624 A1
(43) Pub. Date: Oct. 6, 2022

(54) THIOSTREPTON ANALOGS AND METHODS OF MAKING AND USING SAME

(71) Applicant: YALE UNIVERSITY, New Haven, CT (US)

(72) Inventors: Jonathan Ellman, Guilford, CT (US); Ryan Scamp, New Haven, CT (US); Scott Miller, Woodbridge, CT (US); Edward de Ramon, New Haven, CT (US)

(21) Appl. No.: 17/610,778

(22) PCT Filed: May 13, 2020
(86) PCT No.: PCT/US20/32565

§ 371 Date: Nov. 12, 2021

Related U.S. Application Data
(60) Provisional application No. 62/847,134, filed on May 13, 2019.

(51) Int. Cl. C07K 5/083; C07K 5/103; A61P 31/04

(52) U.S. Cl. CPC C07K 5/0806; C07K 5/101; A61P 31/04; A61K 38/00

(57) ABSTRACT

Provided herein are thiostrepton analogues, such as but not limited to compounds of Formula I, with improved aqueous solubility and antibacterial properties against antibiotic-resistant bacterial strains. The methods of the disclosure further provide a chemoselective way to introduce diverse functionality into peptides or protein comprising a dehydroalanine residue.

### CROSS-REFERENCE TO RELATED APPLICATION

This application claims priority to U.S. Provisional Patent Application Ser. No. 62/847,134 entitled “THIOSTREPTON ANALOGS AND METHODS OF MAKING AND USING SAME,” filed May 13, 2019, the disclosure of which is incorporated herein by reference in its entirety.

### STATEMENT REGARDING FEDERALLY SPONSORED RESEARCH

This invention was made with government support under GM122473 and GM068649 awarded by National Institutes of Health. The government has certain rights in the invention.

### BACKGROUND

Thiostrepton is a highly complex macrocyclic peptide-derived natural with extremely potent activity against Gram-positive bacteria. In fact, thiostrepton is considerably more potent against Gram-positive bacteria than the benchmark antibiotic vancomycin. However, despite the exceptional activity of thiostrepton in vitro, it is not used clinically largely due to its very low aqueous solubility.

Although thiostrepton has been prepared by total synthesis, its high level of structural complexity renders unrealistic the development of improved analogues by total synthesis. As a potential alternative, facile generation of thiostrepton in large quantities by fermentation could be used to prepare thiostrepton analogues either by altering thiostrepton's biosynthesis or by synthetic elaboration. Nonetheless, these methods are severely constrained by the presence of several reactive functionalities in thiostrepton and the compound's propensity towards degradation under standard reaction conditions. Due to these challenges, few thiostrepton analogues have been prepared, and maintaining antibacterial activity of these analogues while significantly improving their aqueous solubility has not be reported.

There is consequently a need for thiostrepton analogues with improved aqueous solubility and antibacterial activity. These analogues should be synthesized chemoselectively without affecting any of the delicate functional groups in thiostrepton. The present invention addresses these needs.

### BRIEF DESCRIPTION OF THE FIGURES

The drawings illustrate generally, by way of example, but not by way of limitation, various embodiments of the present application.

FIG. 1 illustrates a general non-limiting scheme for preparing compounds of Formula I using cobalt-catalyzed C—H amidation of thiostrepton, in accordance with various embodiments.

FIG. 2 illustrates a scheme for preparing compounds of Formula I using cobalt-catalyzed C—H amidation of thiostrepton using a benzaldehyde derivative, followed by imidation, in accordance with various embodiments.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others. The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, triose (e.g., glycerlaldehyde, dihydroxyacetone, and the like), tetrose (e.g., erythrose, threose, eryhtrulose, and the like), pentose (e.g., arabinose, lyxose, ribose, xylose, ribulose, xylulose, ribulose, and the like), hexose (e.g., allose, altrose, glucose, mannose, gulose, idose, galactose, talose, and the like), heptose (e.g., sedoheptulose, mannoheptulose, and the like), and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

As used herein, the term “heteroalkyl” refers to an alkyl group in which one or more of the carbon atoms in the alkyl chain is replaced with a heteroatom. Non-limiting examples of heteroatoms include O, N, S, P, B, and Si.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “standard temperature and pressure” as used herein refers to 20° C. and 101 kPa.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

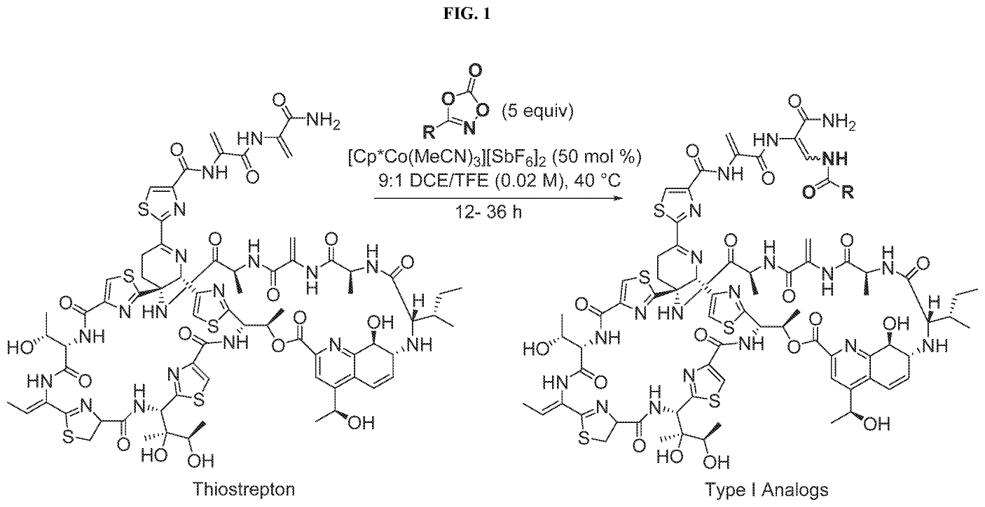

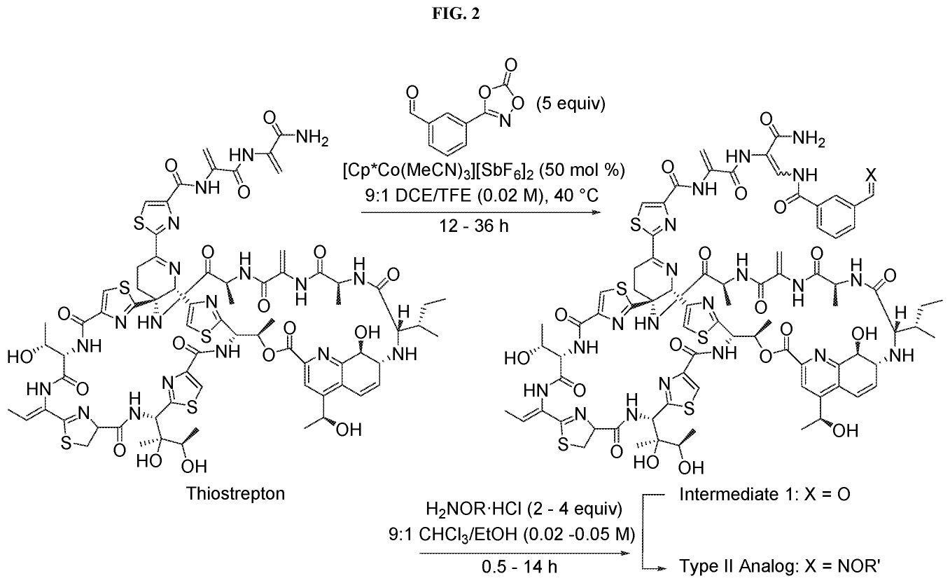

Compounds of Formula I or otherwise described herein can be prepared by the general schemes described herein. The following examples illustrate non-limiting embodiments of the compound(s) described herein and their preparation. Compounds of Formula I can be prepared according to the synthetic schemes illustrated in FIG. 1 and FIG. 2, respectively.

As provided in more detail herein, the preparation, characterization, antibacterial activity and solubility of thiostrepton analogs (FIG. 1 and FIG. 2) is provided. In certain embodiments, the compounds of Formula I have comparable antibacterial activity to thiostrepton. In other embodiments, the compounds of Formula I have improved aqueous solubility over thiostrepton. In yet other embodiments, the compounds of Formula I have improved developability for treatment of bacterial infections over thiostrepton. In yet other embodiments, the compounds of Formula I display unique amide chemical linkage and retain the dehydroamino acid structure of thiostrepton.

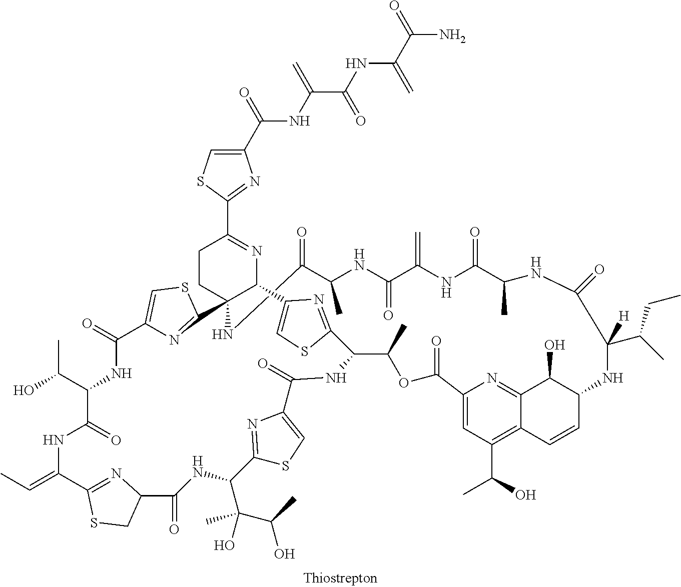 [SMILES: C=C(NC(=O)C(=C)NC(=O)c1csc(C2=N[C@@H]3c4csc(n4)[C@H]4NC(=O)c5csc(n5)[C@H]([C@](C)(O)[C@@H](C)O)NC(=O)C5CSC(=N5)/C(=C/C)NC(=O)[C@H]([C@@H](C)O)NC(=O)c5csc(n5)[C@]3(CC2)NC(=O)[C@H](C)NC(=O)C(=C)NC(=O)[C@H](C)NC(=O)[C@H]([C@@H](C)CC)N[C@@H]2C=Cc3c([C@H](C)O)cc(nc3[C@H]2O)C(=O)O[C@@H]4C)n1)C(N)=O]

As demonstrated herein, transition metal catalyzed C—H functionalization was used for the first time to introduce amide linkage at a specific dehydroalanine site in thiostrepton in good yield and in a single step, while maintaining the dehydro amino acid structure.

Thiostrepton can be reacted with 3-R¹ substituted-1,4,2-dioxazol-5-one

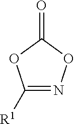

in the presence of a transition metal catalyst as illustrated in FIG. 1. In certain embodiments, R¹ is H, C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents. The dioxazolone reagent can be prepared using methods known in the literature or obtained from commercial sources, as necessary or applicable.

Different transition metal catalysts can be used to introduce these modifications with variable effectiveness. In various embodiments, transition metal catalysts comprise a transition metal M. In various embodiments, M is Ti. In various embodiments, M is V. In various embodiments, M is Cr. In various embodiments, M is Mn. In various embodiments, M is Fe. In various embodiments, M is Co. In various embodiments, M is Ni. In various embodiments, M is Cu. In various embodiments, M is Zn. In various embodiments, M is Zr.

In various embodiments, M is Nb. In various embodiments, M is Mo. In various embodiments, M is Ru. In various embodiments, M is Rh. In various embodiments, M is Pd.

In various embodiments, M is Ag. In various embodiments, M is W. In various embodiments, M is Re. In various embodiments, M is Os. In various embodiments, M is Ir.

In various embodiments, M is Pt. In various embodiments, M is Au. In various embodiments, M is any combinations of the metals contemplated herein. In various embodiments, the transition metal catalyst has the formula [Cp*M(solv)_{d}][SbF₆]ₑ, wherein Cp* is a 1,2,3,4,5-pentamethylcyclopentadienyl (C₅(CH₃)₅⁻) anion optionally substituted by 1 to 5 organic groups, M is a transition metal, solv is an aprotic solvent, and ‘d’ and ‘e’ are independently integers from 1 to 3, wherein the sum of ‘d’ and ‘e’ is not more than 5. In various embodiments, solv is MeCN. In various embodiments, ‘d’ is 3 and ‘e’ is 2.

In certain embodiments, the transition metal catalyst employed for the C—H functionalization step to introduce the amide linkage is [Cp*Co(MeCN)₃][SbF₆]₂ (FIG. 1). This procedure can be employed to produce large quantities of Type I analogs from thiostrepton, which is readily produced by fermentation and is inexpensive. Further, the cobalt-catalyzed C—H amidation step can be used to prepare analogues of any peptide or protein that incorporates at least one dehydroalanine residue.

The amount of catalyst of formula [Cp*M(solv)_{d}][SbF₆]ₑ used in the C—H functionalization step can be from about 1 mol % to about 60 mol %, or about 10 mol % to 60 mol %, or about 20 mol % to about 60 mol %, or about 30 mol % to about 60 mol %, or about 40 mol % to about 60 mol %. In various embodiments, the amount of catalyst used is about 1 mol %, 5 mol %, 10 mol %, 15 mol %, 20 mol %, 25 mol %, 30 mol %, 35 mol %, 40 mol %, 45 mol %, 50 mol %, 55 mol %, or about 60 mol %. In various embodiments, about 50 mol % of [Cp*M(solv)_{d}][SbF₆]ₑ is used.

The solvent used in the reactions of FIG. 1 or FIG. 2 can be a mixture of a non-polar aprotic solvent and a polar protic solvent, or a mixture of a non-polar aprotic solvent and a polar aprotic solvent. Non-limiting examples of non-polar aprotic solvents include chloroform, diethyl ether, pentane, hexane, benzene, toluene, DCE (1,2-dichloroethane), and the like. Non-limiting examples of polar aprotic solvents include DMSO (dimethylsulfoxide), DMF (dimethylformamide), acetone, NMP (N-methyl-2-pyrrolidone), 1,4-dioxane, acetonitrile, HMPA (hexamethylphosphoramide), and the like. Non-limiting examples of polar protic solvents include water, ethanol, methanol, iso-propanol, TFE (2,2,2-trifluoroethanol), and the like.

In various embodiments, the non-polar aprotic solvent is DCE and the polar protic solvent is TFE. In various embodiments, the solvent is a mixture of a non-polar solvent and a polar protic solvent in a ratio of about 10:1, 9:1, 8:1, 5:1, 2:1, 1:1, 1:2, 1:5, 1:8, 1:9, or 1:10. In various embodiments, the solvent is a mixture of DCE and TFE in a DCE:TFE ratio of about 10:1, 9:1, 8:1, 5:1, 2:1, 1:1, 1:2, 1:5, 1:8, 1:9, or 1:10. In one embodiment, the solvent is DCE:TFE in a 9:1 ratio. In various embodiments, the C—H amidation reaction described herein is conducted with an initial thiostrepton concentration of about 0.005 M to about 0.1 M, or about 0.01 M to about 0.05 M. In various embodiments, the reaction described herein is conducted with an initial thiostrepton concentration of about 0.005 M, 0.01 M, 0.02 M, 0.03 M, 0.04 M, 0.05 M, 0.06 M, 0.07 M, 0.08 M, 0.09 M, or 0.1 M.

Type II analogs are prepared in a two-step process (FIG. 2). Cobalt-catalyzed C—H amidation of thiostrepton is performed to give amide 2, wherein R′ comprises a ketone or aldehyde group, such as but not limited to an aromatic ketone or aldehyde group. Oxime formation with an O-alkoxylamine is used to chemoselectively introduce a very large variety of functionality, including sugars, pegylation, amines, acids and alcohol functionality.

In various embodiments, a compound of Formula I, or a salt, solvate, enantiomer, tautomer, or diastereomer thereof, is provided:

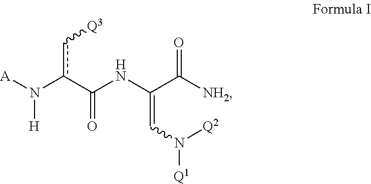

wherein A has the structure:

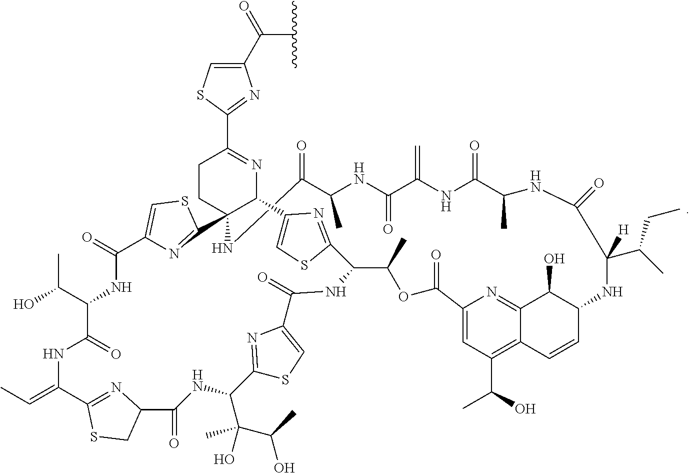 [SMILES: *C(=O)c1csc(C2=N[C@@H]3c4csc(n4)[C@H]4NC(=O)c5csc(n5)[C@H]([C@](C)(O)[C@@H](C)O)NC(=O)C5CSC(=N5)/C(=C/C)NC(=O)[C@H]([C@@H](C)O)NC(=O)c5csc(n5)[C@]3(CC2)NC(=O)[C@H](C)NC(=O)C(=C)NC(=O)[C@H](C)NC(=O)[C@H]([C@@H](C)CC)N[C@@H]2C=Cc3c([C@H](C)O)cc(nc3[C@H]2O)C(=O)O[C@@H]4C)n1]

In the compound of Formula I,
Q¹ is selected from H and (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents;
Q² is hydrogen;
[symbol] is a covalent single bond or a covalent double bond, where
   a) if [symbol] is a covalent double bond then Q³ is H or (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents, provided that if Q³ is H then Q¹ is not H, and if Q³ is not H then Q¹ is H, and
   b) if [symbol] is a covalent single bond then Q³ is NH—(CH₂)ₙ—*, n is an integer from 1 to 10, and the * represents a covalent point of attachment to an open valence on Q¹ to form a ring.

In various embodiments, Q¹ is H. In various embodiments, Q¹ is (C═O)—R¹. In various embodiments, R¹ is optionally substituted C₁₋₁₂ hydrocarbyl. In various embodiments, R¹ is methyl. In various embodiments, R¹ is ethyl. In various embodiments, R¹ is propyl. In various embodiments, R¹ is iso-propyl. In various embodiments, R¹ is butyl. In various embodiments, R¹ is s-butyl. In various embodiments, R¹ is t-butyl. In various embodiments, R¹ is pentyl. In various embodiments, R¹ is hexyl. In various embodiments, R¹ is heptyl. In various embodiments, R¹ is octyl. In various embodiments, R¹ is nonyl. In various embodiments, R¹ is decyl. In various embodiments, R¹ is undecyl. In various embodiments, R¹ is dodecyl. In various embodiments, R¹ is methyl, heptyl, or iso-propyl. The alkyl chain can be substituted on any of the carbon in the chain with OH, OR², NHR², NHR¹R², CO₂R², CO₂H, COR², CR²═N—OR³. In various embodiments, Q¹ is (C═O)—R¹ and R¹ is substituted with at least one substituent selected from the group consisting of OH, OR′, NHR′, NHR′R″, CO₂R′, COR′, CR′═N—OR″, wherein R′ and R″ are each independently H or a C₁₋₆ hydrocarbyl.

In various embodiments, R¹ is optionally substituted C₃₋₁₀ cycloalkyl.

In various embodiments, R¹ is optionally substituted C₃₋₁₀ heterocycloalkyl.

In various embodiments, R¹ is optionally substituted C₃₋₁₀ heteroaryl

In various embodiments, R¹ is optionally substituted C₁₋₁₂ heteroalkyl. In various embodiments, R¹ is —CH₂—[O—CH₂CH₂]ₚ—OCH₃, wherein p is an integer from 0 to 10. In various embodiments, p is 0. In various embodiments, p is 1.

In various embodiments, R is optionally substituted C₆₋₁₀ aryl. In various embodiments, R¹ has the structure

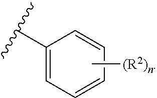

wherein n is an integer from 0 to 5,

wherein each occurrence of R² is independently selected from the group consisting of H, F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, C(═NH)N(R)₂, CR═N—N(R)R³, and CR═N—OR³,

wherein each occurrence of R is independently hydrogen, C₁-C₆ alkyl, or C₃-C₈ cycloalkyl, and

wherein R³ is selected from the group consisting of H, [(CH₂)_{q}—O]ᵣ—CH₃, triose, tetrose, pentose, hexose, heptose, and C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, wherein q and r are independently integers from 1 to 10.

In various embodiments, R² is selected from the group consisting of C(═O)H, C(═O)CH₃, OH, OCH₃, F, Cl, and Br.

In various embodiments, R² is H. In various embodiments, R² is F. In various embodiments, R² is Cl. In various embodiments, R² is Br. In various embodiments, R² is I. In various embodiments, R² is CN. In various embodiments, R² is NO. In various embodiments, R² is NO₂. In various embodiments, R² is ONO₂. In various embodiments, R² is azido. In various embodiments, R² is CF₃. In various embodiments, R² is OCF₃. In various embodiments, R² is OR. In various embodiments, R² is SR. In various embodiments, R² is S(═O)R. In various embodiments, R² is S(═O)₂R. In various embodiments, R² is R. In various embodiments, R² is N(R)₂. In various embodiments, R² is OC(═O)N(R)₂. In various embodiments, R² is S(═O)₂N(R)₂. In various embodiments, R² is SO₃R. In various embodiments, R² is C(═O)R. In various embodiments, R² is C(═O)OR. In various embodiments, R² is OC(═O)R. In various embodiments, R² is C(═O)N(R)₂. In various embodiments, R² is OC(═O)N(R)₂. In various embodiments, R² is (CH₂)₀₋₂N(R)C(═O)R. In various embodiments, R² is N(R)S(═O)₂R. In various embodiments, R² is N(R)C(═O)OR. In various embodiments, R² is N(R)C(═O)R. In various embodiments, R² is N(R)C(═O)N(R)₂. In various embodiments, R² is C(═NH)N(R)₂. In various embodiments, R² is CR═N—N(R)R³. In various embodiments, R² is CR═N—OR³.

In various embodiments, R is hydrogen. In various embodiments, R is C₁-C₆ alkyl. In various embodiments, R is C₃-C₈ cycloalkyl.

In various embodiments, R³ is H. In various embodiments, R³ is [(CH₂)_{q}—O]ᵣ—CH₃. In various embodiments, R³ is triose. In various embodiments, R³ is tetrose. In various embodiments, R³ is pentose. In various embodiments, R³ is hexose. In various embodiments, R³ is heptose. In various embodiments, R³ is C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, wherein q and r are independently integers from 1 to 10.

In various embodiments, R¹ has the structure

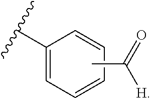

In various embodiments, R¹ has the structure

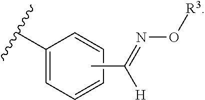

In various embodiments, q is 2 and r is 3. In various embodiments, R³ is glucose. The glucose can be in a linear form or cyclic form, and when glucose is in the cyclic form, it can be in the α- or β-configuration. R³ can, in some embodiments, be either the C₁ or C₆ carbon in glucose.

In various embodiments, [symbol] is a covalent single bond, R¹ has the structure

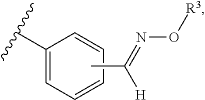

and Q³ is covalently bonded to Q¹ at the R³ position to form a ring.

In various embodiments, the compound of Formula I has an aqueous solubility of greater than about 3 μg/mL at a pH of about 6.9, 7, 7.1, 7.2, 7.3, or 7.4. In various embodiments, the compound of Formula I has an aqueous solubility of greater than about 3 μg/mL at a pH of about 6.9 to about 7.4 in about 1 mM to about 50 mM aqueous MOPS (3-(N-morpholino)propanesulfonic acid) buffer.

In various embodiments, the compound is selected from the group consisting of:

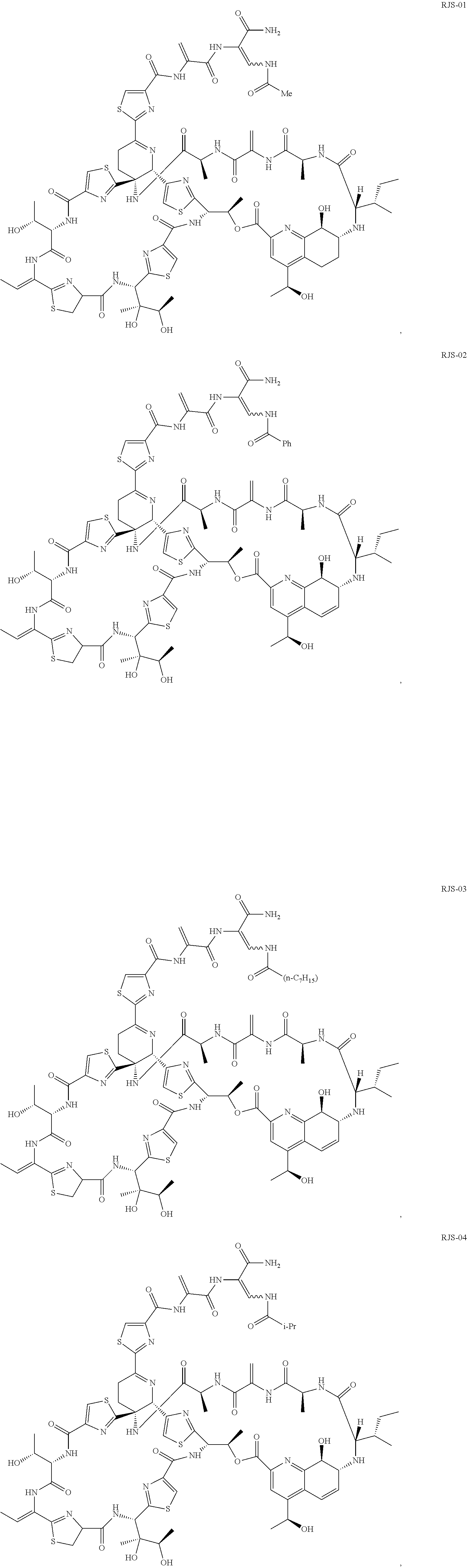

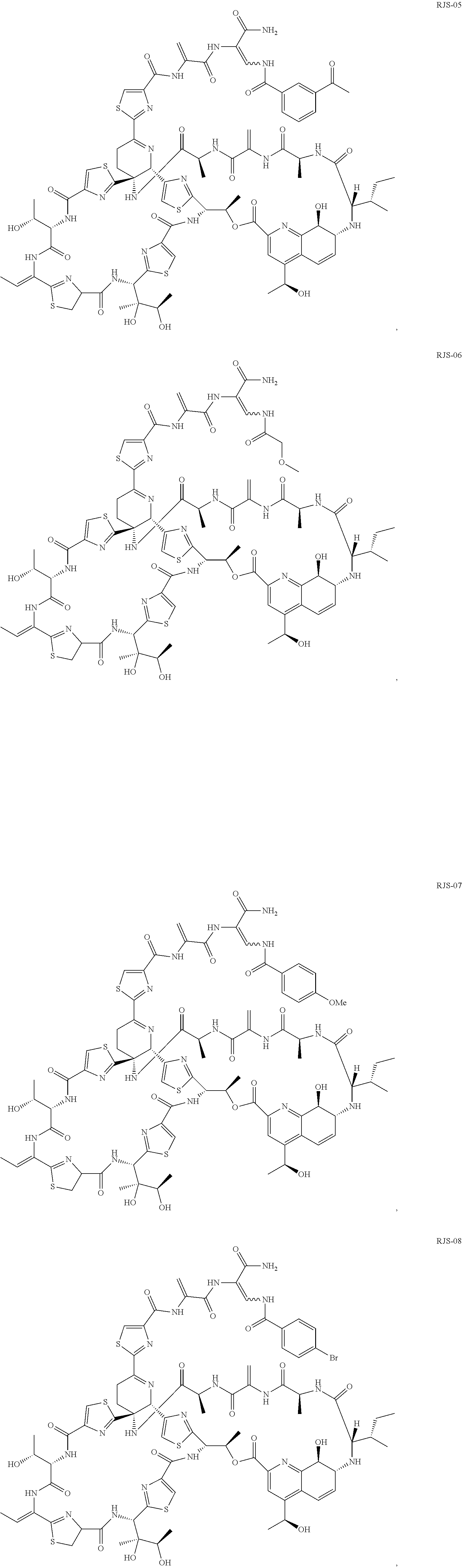

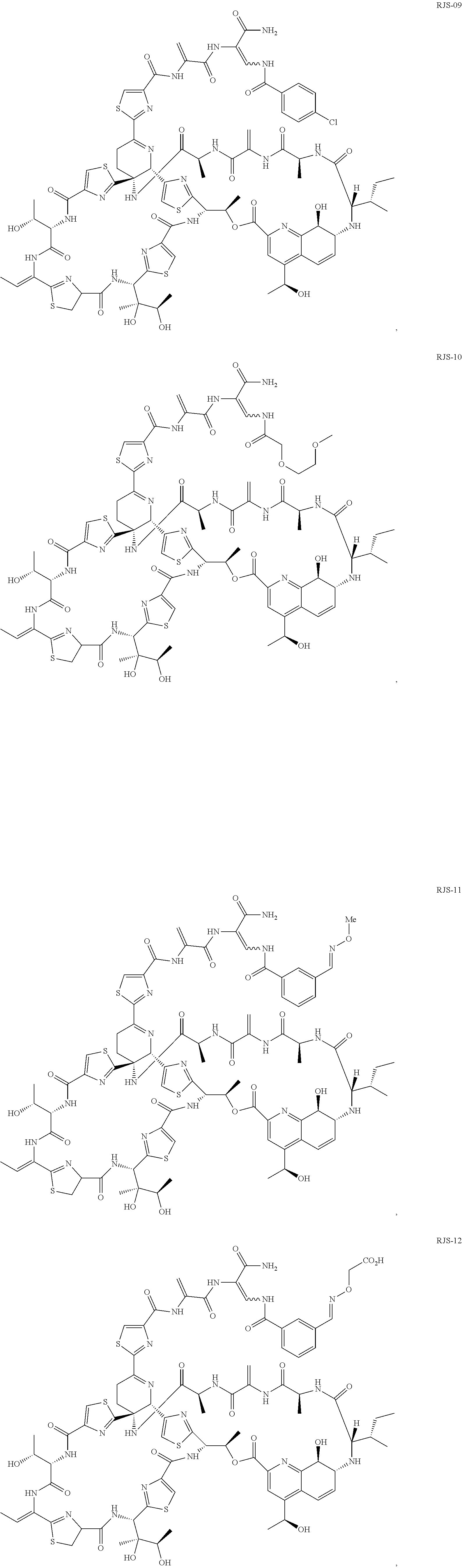

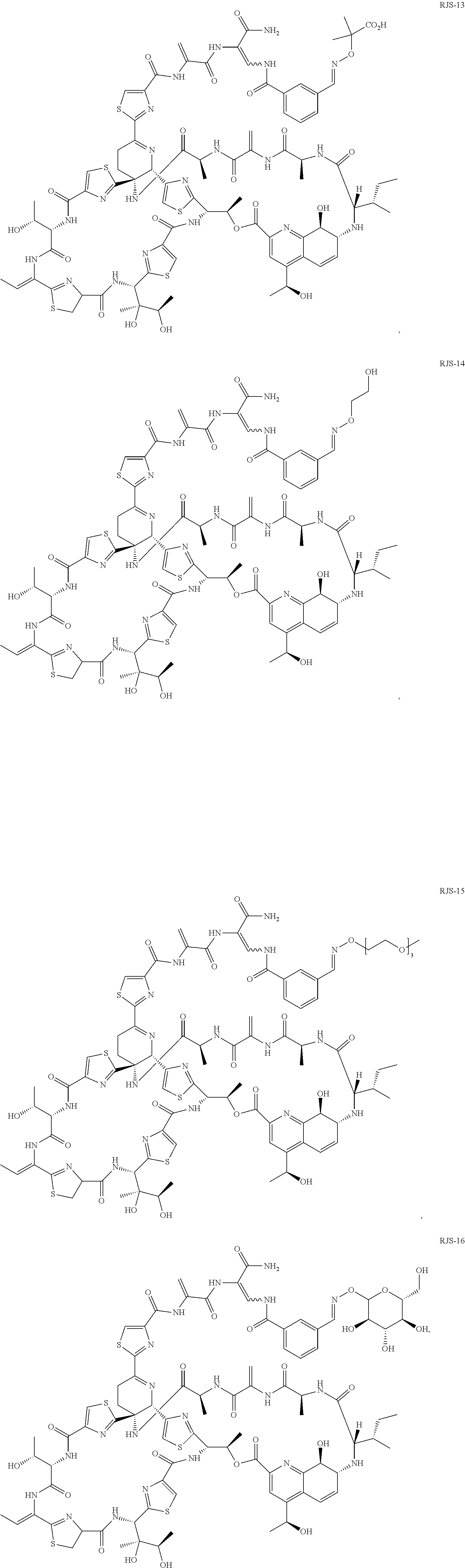

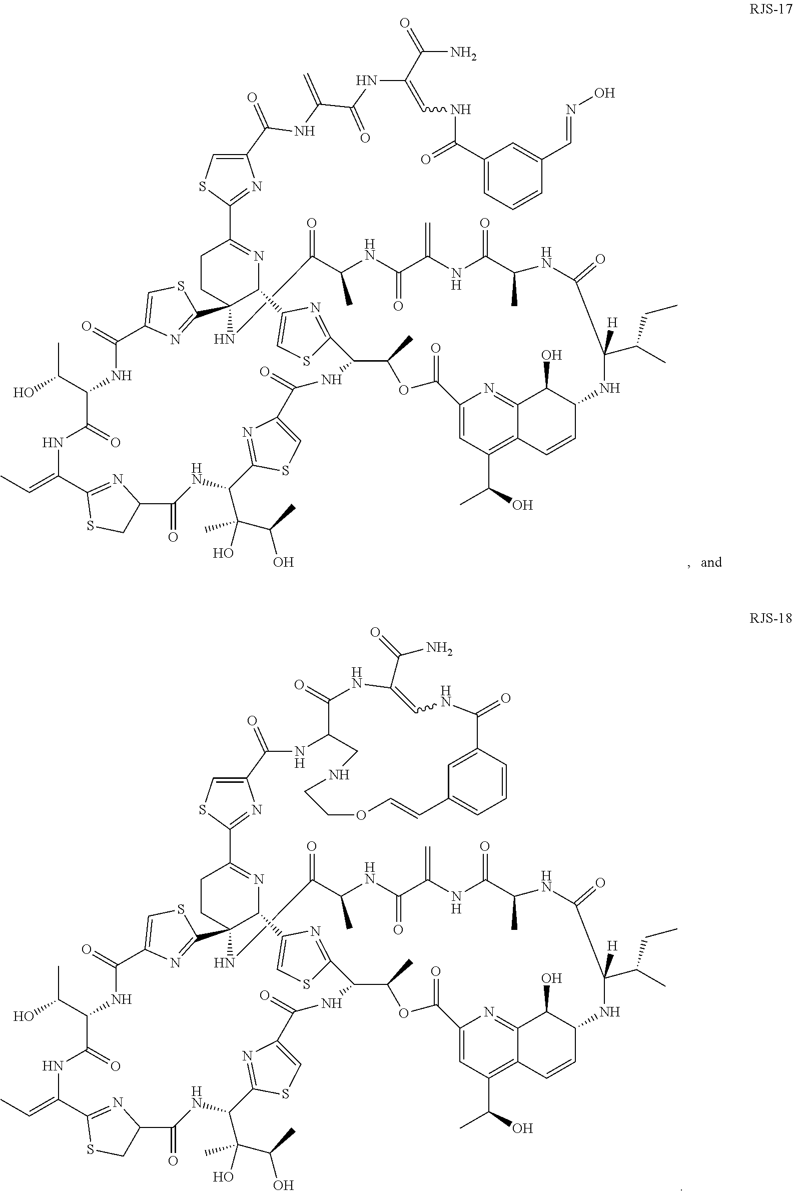

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

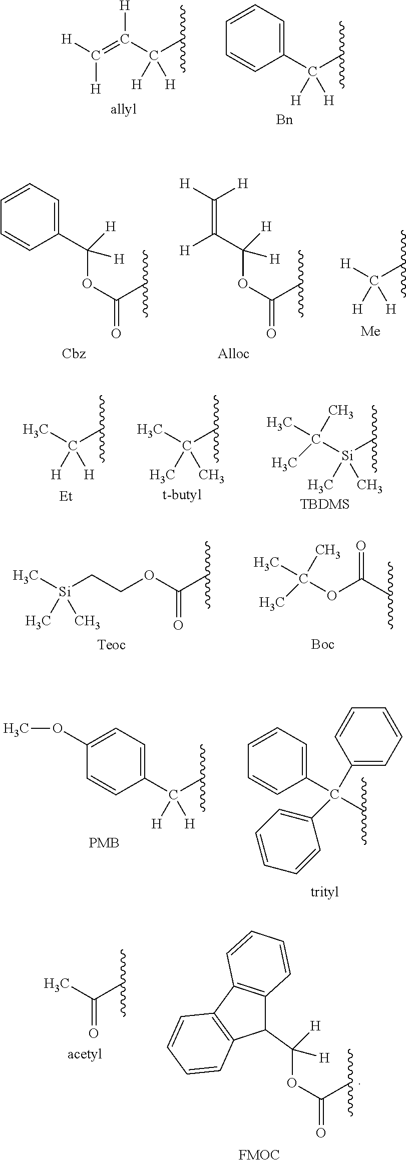

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment and Use

A method of killing, preventing growth, and/or disinfecting bacteria includes, in various embodiments, contacting a bacterial population with a compound of Formula I, wherein the bacterial population is killed and/or disinfected, and/or their growth is prevented, after coming into contact with the compound of Formula I.

The compounds of Formula I have anti-bacterial properties, and are able to kill at least, or greater than about 95%, 98%, 99%, 99.1%, 99.2%, 99.3%, 99.4%, 99.5%, 99.6%, 99.7%, 99.8%, 99.9%, 99.99%, 99.999%, or 99.9999% of bacteria that come in contact with or are exposed to the compounds of Formula I. In various embodiments, the bacteria that are killed are pathogenic bacteria that cause deleterious infections and/or diseases in mammals. In various embodiments, the bacteria are Gram-positive. In various embodiments, the mammal is a cat, dog, human, sheep, horse, mouse, rabbit, rat, cow, goat, pig, and the like. The term “kill” as used herein means that the bacteria are no longer able to exhibit or produce any harmful effect to or in a living organism, and/or that the bacteria are unable to cause further infection, and/or the bacteria cease to live.

The types of bacteria that can be killed by the compounds of the invention is not particularly limited; Non-limiting examples of bacteria genera that are killed when exposed to compounds of Formula I include Bacillus, Clostridium, Corynebacterium, Enterococcus, Listeria, Mycobacterium, Mycoplasma, Staphylococcus, Streptococcus, and Ureaplasma.

Additionally, the compounds of Formula I can kill any species of bacteria in the aforementioned genera that are known to infect humans or other mammals. In various embodiments, the compounds of Formula I kill VRE, PISP, and/or MRSA. In various embodiments, the bacterial population includes methicillin-resistant Staphylococcus aureus (MRSA), vancomycin-resistant Enterococcus faecalis (VRE), vancomycin-sensitive Enterococcus faecalis (VSE), penicillin-resistant S. pneumoniae (PISP), and/or a combination thereof.

In various embodiments, the compounds of Formula I have a minimum inhibitory concentration (MIC) with respect to any of the bacteria described herein of about 0.01 μg/mL to about 5 μg/mL, about 0.01 μg/mL to about 3 μg/mL, about 0.01 μg/mL to about 2 μg/mL, or about 0.01 μg/mL to about 1 μg/mL. In various embodiments, the compounds of Formula I have a MIC with respect to any of the bacteria described herein of at least, greater than, or less than about 0.01 μg/mL, 0.015 μg/mL, 0.025 μg/mL, 0.035 μg/mL, 0.045 μg/mL, 0.055 μg/mL, 0.065 μg/mL, 0.075 μg/mL, 0.085 μg/mL, 0.095 μg/mL, 0.1 μg/mL, 0.15 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2.0 μg/mL, 2.5 μg/mL, 3 μg/mL, 3.5 μg/mL, 4 μg/mL, 4.5 μg/mL, or 5 μg/mL.

In various embodiments, the compounds of Formula I can be used to treat bacterial infections present in the body or on the skin of a mammal. Non-limiting examples of bacterial infections include bacterial vaginosis, bacterial meningitis I, bacterial pneumonia, bacterial upper respiratory infections, ear infections, eye infections, skin infections, thrush, urinary tract infection, bacterial gastroenteritis, impetigo, erysipelas, and cellulitis. A method of treating a bacterial infection in a subject includes administering to the subject a therapeutically effective amount of a composition comprising at least one pharmaceutically acceptable excipient and a compound of Formula I. The subject is, in various embodiments, a human.

In various embodiments, the compounds of Formula I can be used to disinfect non-living objects, including non-living objects or surfaces made from metals, ceramics, glass, wood, fabrics, rubber, plastic, polymers, and composite materials made from any combination of the foregoing, and combinations thereof. In various embodiments, the bacterial population is present on or in a non-living object. As used herein, the term “disinfect” means at least about 95%, 98%, 99%, 99.1%, 99.2%, 99.3%, 99.4%, 99.5%, 99.6%, 99.7%, 99.8%, 99.9%, 99.99%, 99.999%, or 99.9999% of bacteria on or in a non-living object that come into contact with or are exposed to the compounds of Formula I are no longer able to exhibit or produce any harmful effect to or in a living organism, and/or that the bacteria are unable to cause further infection, and/or the bacteria cease to live. In various embodiments, the bacteria disinfected on or from a non-living surface are pathogenic bacteria that can cause deleterious infections and/or diseases in mammals.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that kills, prevents growth, and/or disinfects bacteria.

In certain embodiments, administering the compound(s) described herein to the subject allows for administering a lower dose of the additional therapeutic agent as compared to the dose of the additional therapeutic agent alone that is required to achieve similar results in killing and/or disinfecting bacteria, and/or prevents their growth, in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for killing, preventing growth, or disinfecting bacteria. These additional therapeutic agents may comprise compounds that are commercially available or synthetically accessible to those skilled in the art. These additional therapeutic agents are known to treat, prevent, or reduce the symptoms, of a bacterial infection.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a bacterial infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to kill and/or disinfect bacteria, and/or prevent their growth, in the patient. An effective amount of the therapeutic compound necessary to achieve a therapeutic effect may vary according to factors such as the state of the disease or disorder in the patient; the age, sex, and weight of the patient; and the ability of the therapeutic compound to kill and/or disinfect bacteria, and/or prevent their growth, in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a bacterial infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a bacterial infection in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### General Information

All reactions were initially set up in a N₂ atmosphere glove box. Thiostrepton and [Cp*Co(MeCN)₃][SbF₆]₂ were stored in the glove box at 0° C. in light-proof vials to ensure longevity. Solvents were sparged with N₂ and passed through a silica column prior to introduction to the glove box. Solvents used in purification were HPLC grade and used as received from common chemical suppliers. Thiostrepton was purchased from EMD Millipore.

Analytical UPLC-MS was conducted using Waters Acquity UPLC® BEH C8 (1.7 μm, 2.1×100 mm) column on a Waters XEVO instrument equipped with ESI, a QToF mass spectrometer, and a photodiode array detector. Analytical, Chiral HPLC was conducted using an Agilent 1100 series chromatograph equipped with a photodiode array detector (210, 220, 230, and 254 nm). A SymmetryPrep C8 7 m 19×300 mm reverse-phase column was employed for all separations. Column temperatures were unregulated. Preparative HPLC was conducted using a Shimadzu Prominence HPLC system equipped with a photodiode array detector (210 nm). Column temperature was unregulated. High resolution electrospray ionization mass spectrometry was acquired on a Waters Xevo Qtof high-resolution mass spectrometer with an Acquity UPLC BEH C18 1.7 μm-2.1×50 mm column. Flash Chromatography was conducted using a Biotage Isolera One purification system using either silica columns (normal phase) or C-18 silica columns (reverse phase). NMR solvents were used as received. NMR spectra of thiostrepton derivatives were acquired using a 600 MHz Agilent DD2 or 800 MHz Agilent NMR spectrometer with 3:1 CDCl₃ to MeOD as solvent. NMR data were processed with MestraNova. All spectra were referenced to residual solvent peaks.

### Example 1: Method A—C—H Amidation of Thiostrepton

An oven-dried microwave vial (˜10 mL) containing a ¼″ stir bar was taken into a N₂-pressurized glove box. Thiostrepton (83.2 mg, 0.050 mmol), [Cp*Co(MeCN)₃][SbF₆]₂ (19.7 mg, 0.025 mmol, 50 mol %), and the appropriate dioxazolone (0.1 mmol, 2 equiv) were added to the vial in the listed sequence. A 9:1 mixture of DCE/TFE (2.5 mL) was added, and the vial was capped prior to removal from the glove box. The mixture was stirred at 40° C. in the absence of light with UPLC monitoring of the reaction. After thiostrepton consumption ceased, a final crude UPLC yield was determined from a sample prepared from a 10 μL aliquot of the reaction solution diluted with 500 μL of 37 μg/mL of N-Boc aniline in 7:3 MeCN/H₂O. Effective separation was achieved with a 10 min isocratic gradient from 20-80% MeCN/H2O with 0.1% formic acid. The remaining reaction mixture was then cooled to room temperature, uncapped, and concentrated at room temperature. Reverse-phase purification (60-100% MeOH in H₂O with 0.1% formic acid) on a Biotage Isolera One purification system equipped with a C18 column (30 g) to remove the Co catalyst from the crude reaction mixture. Preparative HPLC (H₂O-MeCN gradient with 0.1% formic acid) was used to separate the reaction mixtures and isolate the major products of each for characterization.

### Example 2: Method B—Oxime Formations

Amidation with 3-(3-formylphenyl)-1,4,2-dioxazolone was performed according to method A. After catalyst removal on the Biotage instrument (60-100% MeOH—H₂O), the fractions pertaining to the thiostrepton derivative mixture were combined and concentrated on a rotary evaporator. The crude amide mixture was dissolved in a 9:1 mixture of CHCl₃/EtOH (2.5 mL). The O-alkyl hydroxylamine salt (2 equiv relative to thiostrepton) was added and the reaction mixture was stirred with UPLC monitoring. For neutral O-alkyl hydroxylamines, 1 equiv of 1 N HCl was added to form the hydrochloride salt in situ. The oxime mixture was partitioned with 10 mL of brine and extracted with 3×5 mL of CHCl₃. The organic layers were combined and concentrated. A final crude UPLC yield was determined from a sample prepared from a 10 μL aliquot of the reaction solution diluted with 500 μL of 37 μg/mL of N-Boc aniline in 7:3 MeCN/H₂O. Effective separation was achieved with a 10 min isocratic gradient from 20-80% MeCN/H₂O with 0.1% formic acid. Preparative HPLC (H₂O-MeCN gradient with 0.1% formic acid) was used to separate the remaining reaction mixture and isolate the major product for characterization.

### Example 3

Table 1 lists the yields and molecular ions for compounds of Formula I.

TABLE 1
Yields and molecular ions for thiostrepton analogs.

| | % UPLC Yield | ESI Molecular Ion |
|---|---|---|
| Compound | (regioisomeric ratio) | (m/z) [M + 2H]2+ |
| RJS-01 | 50 (>20:1)^{a} | 861.2647 |
| RJS-02 | 63 (5.3:1)^{a} | 892.2726 |
| RJS-03 | 39 (3.3:1)^{a} | 903.8156 |
| RJS-04 | 36 (>20:1)^{a} | 875.2725 |
| RJS-05 | 63 (16:4.2:1)^{a} | 913.2778 |
| RJS-06 | 40 (1.7:1)^{a} | 876.2700 |
| RJS-07 | 41 (4.1:1)^{a} | 907.2778 |
| RJS-08 | 60 (10:1)^{a} | 931.2778 |
| RJS-09 | 44 (3.4:1)^{a} | 909.2531 |
| RJS-10 | 47 (4.2:1)^{a} | 898.2831 |
| RJS-11 | 35 (7.5:2.2:1)^{b} | 920.7833 |
| RJS-12 | 31 (4.5:2.3:1)^{b} | 942.7782 |
| RJS-13 | 42 (2.3:1)^{b} | 956.7939 |
| RJS-14 | 50 (10.3:5.6:1)^{b} | 935.7886 |
| RJS-15 | 59 (4.2:1.9:1)^{b} | 986.8226 |
| RJS-16 | 52 (2.7:1)^{b} | 994.8019 |
| RJS-17 | 20 (major isomer)^{b,c} | 913.7755 |
| RJS-18 | 33 (6.5:3.3:1)^{b} | 935.2966 |

^{a}Yield over one step from thiostrepton as starting material based on crude UPLC traces with N-(tert-butoxycarbonyl)aniline as internal standard.
^{b}Yield over two steps from thiostrepton as starting material based on crude UPLC traces with N-(tert-butoxycarbonyl)aniline as internal standard.
^{c}Minor isomers not determined owing to overlap with starting material.

In Table 1, the major isomer has the structure of Formula Ia:

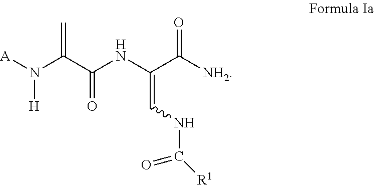

The minor isomer in Table 1 has the structure of Formula Ib:

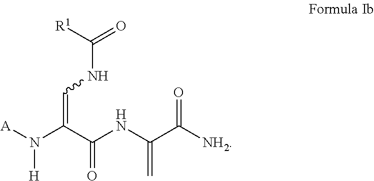

The E:Z ratio in the major or minor isomer can be an E:Z ratio of at least, greater than, or less than about 100:1, 95:1, 90:1, 80:1, 70:1, 60:1, 50:1, 40:1, 30:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:95, or 1:100. In Table 1, the major isomer is present in the largest amount and is represented by the larger of the two numbers in the ratio.

### Example 4: Aqueous Solubility Analysis

A 2 dram vial was loaded with ˜0.5 mg of a thiostrepton derivative and suspended in ˜4 mL of aqueous 10 mM MOPS buffer (pKₐ 7.2). The suspension was stirred vigorously for 24 h to ensure equilibration, then filtered through a 0.22 m centrifuge filtration unit. A 3.0 mL aliquot was concentrated, and the dried residue was diluted with 0.30 mL of 1:1 1,4-dioxane/H₂O (10× original concentration necessitated by detection limits). The total UV absorbance (200-300 nm) was measured in triplicate by UPLC runs with a photodiode array detector. A calibration curve for each compound was created using data points from 3 trials each of concentrations in 1:1 1,4-dioxane/H₂O ranging from 800-25 ag/mL. These concentrations were plotted against their respective average absorbances. The experimental concentration of each compound was calculated by dividing the found μg/mL value by the residue concentration factor of 10 to yield the actual solubility value. Determined solubilities are reported in Table 2.

TABLE 2
Solubility Data for Thiostrepton Derivatives

| Compound | Solubility pH 7.2 (μg/mL)* |
|---|---|
| Thiostrepton | 3.0 (±0.2) |
| RJS-01 | 83 (±5) |
| RJS-02 | <1.0 |
| RJS-03 | <1.0 |
| RJS-04 | 16.2 (±0.6) |
| RJS-05 | 1.1 (±0.2) |
| RJS-06 | 28 (±4) |
| RJS-07 | ND |
| RJS-08 | ND |
| RJS-09 | <1.0 |
| RJS-10 | 19 (±1) |
| RJS-11 | <1.0 |
| RJS-12 | 20 (±1) |
| RJS-13 | ND |
| RJS-14 | 1.4 (±0.4) |
| RJS-15 | 4.3 (±0.4) |
| RJS-16 | 11 (±1) |
| RJS-17 | <1.0 |
| RJS-18 | ND |

*Solubilities after 24 hours in 10 mM aqueous MOPS buffer, pH 7.2.
ND = Not determined.
MOPS = 3-(N-morpholino)propanesulfonic acid.

### Biological Testing

### Test Agents and Comparators

Twenty-four test agents were shipped to Micromyx and stored at −20° C. until assayed. The compounds were dissolved in 100% DMSO at 6.46 mg/mL prior to testing. Comparator drugs were supplied by Micromyx and were handled as described below:

| | | | Stock | |
|---|---|---|---|---|
| Comparator | | | Concentration | |
| Drug | Supplier | Lot. No. | (μg/mL) | Solvent |
| Ceftazidime | Sigma Aldrich | 117M4826V | 3,232 | dH₂O |
| Vancomycin | Sigma Aldrich | 080M1341V | 3,232 | dH₂O |

### Test Organisms

The test organisms for the assay were recent clinical isolates or reference strains acquired from the American Type Culture Collection (ATCC; Manassas, Va.). Upon receipt, the isolates were streaked onto Trypticase Soy Agar II+5% sheep blood (TSAB). Colonies were harvested from the plates and a cell suspension was prepared in Trypticase Soy Broth containing cryoprotectant. Aliquots were then frozen at −80° C. Prior to the assay, the frozen seeds of all bacterial isolates to be tested were thawed and streaked for isolation onto TSAB (BD/BBL, Sparks, Md.; Lot No. 9011894), and were incubated overnight at 35° C.

### Test Media

The test medium for all isolates was cation-adjusted Mueller Hinton broth (CAMHB; BD/BBL; Lot No. 8190586). CAMHB (Cation Adjusted Mueller Hinton Broth) was supplemented with 3% (v/v) lysed horse blood (LHB; Hemostat, Dixon, Calif.; Lot No. 474990) for the testing of streptococci.

### Minimal Inhibitory Concentration (MIC) Assay

MIC values were determined using a broth microdilution method as recommended by CLSI. Automated liquid handlers (Multidrop 384, Labsystems, Helsinki, Finland; Biomek 2000 and Biomek F/X, Beckman Coulter, Fullerton Calif.) were used to conduct serial dilutions and make liquid transfers.

The wells of standard 96-well microdilution plates (Costar 3795, Corning Inc., Corning, N.Y.) were filled with 150 μL of the appropriate diluent in columns 2-12 on the Multidrop 384 (DMSO for the thiostrepton analogs, deionized H₂O for vancomycin and ceftazidime). These plates were used to prepare the drug “mother plate” which provided the serial drug dilutions for the replicate “daughter plates”. The compounds (300 μL) were dispensed at 101× the highest concentration to be tested into the appropriate row of the “mother plate”. The Biomek 2000 was used to transfer 150 μL of each stock solution from the wells in Column 1 of the mother plate to make ten subsequent 2-fold serial dilutions. The wells of Column 12 contained no drug and served as the organism growth control wells. The daughter plates were loaded with 190 μL of test medium using the Multidrop 384.

The daughter plates were prepared on the Biomek F/X instrument which transferred 2 L of drug solution from each well of the mother plate to each corresponding well of each daughter plate in a single step. The wells of the daughter plates ultimately contained 190 μL of media, 2 μL of drug solution, and 10 μL of inoculum prepared as described below. The final concentration of DMSO (if used as a solvent) in the test well was 1%.

A standardized inoculum of each organism was prepared per CLSI methods. A suspension of organisms from a fresh plate was prepared in CAMHB to equal the turbidity of a 0.5 McFarland standard. The suspensions were diluted 1:20 in CAMHB, or in the case of streptococci CAMHB with LHB. The inoculum for each organism was dispensed into sterile reservoirs divided by length (Beckman Coulter), and the Biomek 2000 was used to inoculate the plates. Daughter plates were placed on the Biomek 2000 work surface reversed so that inoculation took place from low to high drug concentration. The Biomek 2000 delivered 10 μL of standardized inoculum into each well. These dilutions yielded a final cell concentration in the daughter plates of approximately 5.0×10⁵ CFU/mL.

Plates were stacked 3 high, covered with a lid on the top plate, placed in plastic bags, and incubated at 35° C. for 16-20 hr for all organisms except streptococci which were incubated for 20-24 hr. Following incubation, the microplates were removed from the incubator and viewed from the bottom using a plate viewer. The MIC was read and recorded as the lowest concentration of drug that inhibited visible growth of the organism. Vancomycin MICs for staphylococci and enterococci were read after 24 hr of incubation per CLSI. An un-inoculated solubility control plate was observed for evidence of drug precipitation.

### Results

Susceptibility testing results are shown in Table 3. Results for vancomycin and ceftazidime were within the established CLSI quality control ranges for the relevant organisms, thus validating the assay. There was distinct precipitation noted for the thiostrepton parent molecule and analogs RJS-4, -8, -9, -10, -11 at 64 μg/mL in CAMHB and for thiostrepton and analogs RJS-8, -16, -17, and -18 at 64 μg/mL in CAMHB in LHB. In no instance did precipitation interfere with the interpretation of the MIC endpoints during the study.

None of the 18 thiostrepton analogs or the parent thiostrepton molecule were active against E. coli ATCC 25922 at the highest concentration tested (64 μg/mL).

Against S. aureus isolates, thiostrepton had MIC values of 0.06-0.12 μg/mL. The most potent analogs against S. aureus had MIC values of 0.25-0.5 μg/mL (RJS-4, RJS-9, RJS-11, RJS-14, and RJS-17) and there were several analogs with MIC values of 1-2 μg/mL. RJS-1 had MIC values of 2-4 μg/mL and RJS-16 was the least active analog against S. aureus with MIC values of 16 μg/mL. The activity of thiostrepton and its analogs was not impacted by methicillin resistance among S. aureus as MIC values against MRSA were identical or within 2-fold of those observed with MSSA.

Similar activity was observed against E. faecalis and E. faecium for thiostrepton and its analogs. Thiostrepton had MICs of 0.06-0.12 μg/mL. Most analogs were active with MIC values of 0.5-1 μg/mL across the evaluated enterococci but two had more potent activity: RJS-9 with MIC values of 0.12-0.25 μg/mL and RJS-11 with MIC values of 0.25 μg/mL excluding one isolate with an MIC of 0.5 μg/mL. As with S. aureus, lower activity was observed with RJS-1 (MIC values of 2-4 μg/mL) and RJS-16 (MIC values of 8-16 μg/mL). The activity of thiostrepton and its analogs was not impacted by vancomycin resistance among enterococci as VRE MIC values were comparable to VSE MIC values.

The most potent activity for thiostrepton and its analogs was observed against the evaluated S. pneumoniae and S. pyogenes isolates. MIC values of ≤0.001 μg/mL were observed for thiostrepton and analogs RJS-12, with MIC values of ≤0.001-2 observed for analogs RJS-9 and RJS-10. The remaining analogs had MIC values of 0.002-0.015 μg/mL with the exception of RJS-18 which had MIC values of 0.03 μg/mL, RJS-1 which had MIC values of 0.06 μg/mL, and RJS-16 which had MIC values of 0.25 μg/mL.

In summary, thiostrepton and its analogs had potent activity against the evaluated Gram-positive cocci and were most potent against streptococci. RJS-9 and RJS-11 appeared to have the most activity among the evaluated analogs while RJS-1 and -16 appeared to have the least activity among the evaluated analogs. Resistance to methicillin among S. aureus and vancomycin among enterococci did not have any apparent impact on the activity of thiostrepton or its analogs.

TABLE 3
Activity of thiostrepton and compounds of Formula I against Gram-positive pathogens.

| | | MIC (μg/mL) | | | | | | | | |
|---|---|---|---|---|---|---|---|---|---|---|
| | | S. aureus | S. aureus | E. faecalis | | | | | | |
| | | ATCC | MMX | ATCC | E. faecalis | E. faecium | E. faecium | S. pneumoniae | | |
| | | 29213 | 8088 | 29212 | MMX 486 | MMX 708 | MMX 485 | ATCC 49619 | S. pyogenes | E. coli |
| Drug | Valiant | MSSA | MRSA | VSE | VRE | VSE | VRE | PISP | ATCC 19615 | ATCC 25922 |
| Thiostrepton | N/A | 0.06 | 0.12 | 0.12 | 0.12 | 0.12 | 0.06 | <0.001 | <0.001 | >64 |
| RJS-01 | Me amide | 2 | 4 | 4 | 4 | 4 | 2 | 0.06 | 0.06 | >64 |
| RJS-02 | Ph amide | 1 | 2 | 1 | 1 | 0.5 | 0.5 | 0.008 | 0.002 | >64 |
| RJS-03 | Heptyl | 1 | 2 | 1 | 1 | 1 | 0.5 | 0.004 | 0.008 | >16 |
| | amide | | | | | | | | | |
| RJS-04 | iPr amide | 0.5 | 0.5 | 0.5 | 1 | 1 | 0.25 | 0.004 | 0.004 | >64 |
| RJS-05 | m-AcPh | 1 | 2 | 1 | 1 | 1 | 0.5 | 0.004 | 0.004 | >64 |
| | amide | | | | | | | | | |
| RJS-06 | MOM | 0.5 | 1 | 1 | 1 | 1 | 0.5 | 0.015 | 0.008 | >64 |
| | amide | | | | | | | | | |
| RJS-07 | p-OMePh | 1 | 2 | 2 | 1 | 1 | 1 | 0.004 | 0.004 | >64 |
| | amide | | | | | | | | | |
| RJS-08 | p-BrPh | 1 | 1 | 0.5 | 0.5 | 0.5 | 0.5 | <0.001 | 0.002 | >64 |
| | amide | | | | | | | | | |
| RJS-09 | p-C1Ph | 0.25 | 0.5 | 0.25 | 0.25 | 0.25 | 0.12 | <0.001 | 0.002 | >64 |
| | amide | | | | | | | | | |
| RJS-10 | PEG-1 | 1 | 1 | 1 | 1 | 1 | 1 | 0.015 | 0.015 | >64 |
| | amide | | | | | | | | | |
| RJS-11 | Me oxime | 0.5 | 0.5 | 0.5 | 0.25 | 0.25 | 0.25 | <0.001 | <0.001 | >64 |
| RJS-12 | AcOH | 1 | 1 | 2 | 1 | 2 | 1 | 0.015 | 0.015 | >64 |
| | oxime | | | | | | | | | |
| RJS-13 | MeAcOH | 1 | 1 | 2 | 1 | 1 | 1 | 0.015 | 0.015 | >64 |
| | oxime | | | | | | | | | |
| RJS-14 | EtOH | 0.5 | 0.5 | 0.5 | 0.5 | 1 | 0.5 | 0.004 | 0.004 | >64 |
| | oxime | | | | | | | | | |
| RJS-15 | PEG-3 | 0.5 | 1 | 1 | 0.5 | 1 | 0.5 | 0.002 | 0.004 | >64 |
| | oxime | | | | | | | | | |
| RJS-16 | 13-D-Glu | 16 | 16 | 16 | 16 | 16 | 8 | 0.25 | 0.25 | >64 |
| | oxime | | | | | | | | | |
| RJS-17 | hHydroxime | 0.5 | 0.5 | 1 | 0.5 | 0.5 | 0.5 | 0.002 | 0.004 | >64 |
| RJS-18 | EtNH2 | 2 | 2 | 2 | 2 | 2 | 1 | 0.03 | 0.03 | >64 |
| | oxime | | | | | | | | | |
| Vancomycin | N/A | 1 (0.5-2)^{a} | 1 | 2 (1-4)^{a} | >32 | 0.25 | >32 | 0.12 (0.12-0.5)^{a} | 0.25 | >64 |
| Ceftazidime | N/A | 8 (4-16)^{a} | >32 | >32 | >32 | >32 | >32 | 0.5 | 0.12 | 0.25 (0.06-0.5)^{a} |

^{a}CLSI quality control range shown in parentheses
MRSA = methacilin-resistant S. aureus,
VRE = vancomycin-resistant enterococci,
PISP = penicillin-resistant S. pneumoniae

### Enumerated Embodiments

The following exemplary embodiments are provided, the numbering of which is not to be construed as designating levels of importance:

Embodiment 1 provides a compound of Formula I, or a salt, solvate, enantiomer, tautomer, or diastereomer thereof:

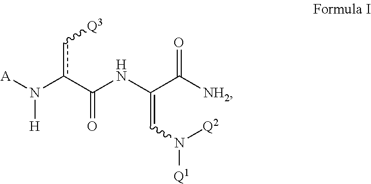

wherein A has the structure:

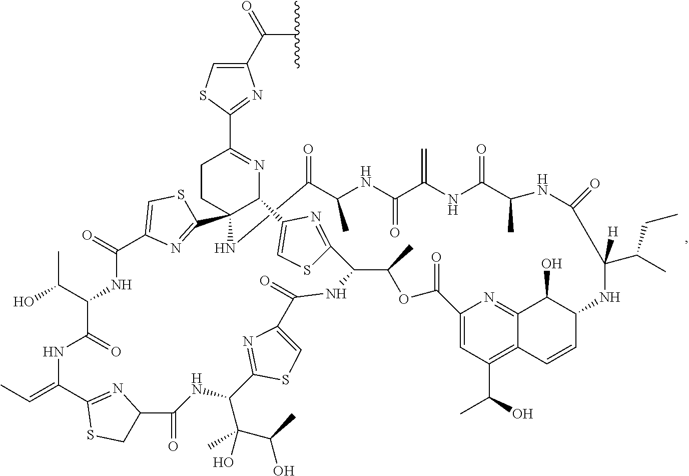

wherein:

Q¹ is H or (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents;

Q² is H;

[symbol] is a covalent single bond or a covalent double bond, wherein
a) if [symbol] is a covalent double bond then Q³ is H or (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents, provided that if Q³ is H then Q is not H, and if Q³ is not H then Q¹ is H, and
b) if [symbol] is a covalent single bond then Q is NH—(CH₂)ₙ—*, n is an integer from 1 to 10, and the * represents a covalent point of attachment to an open valence on Q¹ to form a ring.

Embodiment 2 provides the compound of embodiment 1, wherein Q¹ is (C═O)—R¹.

Embodiment 3 provides the compound of any one of embodiments 1-2, wherein R¹ is C₁₋₁₂ hydrocarbyl.

Embodiment 4 provides the compound of any one of embodiments 1-3, wherein Q¹ is (C═O)—R¹ and R¹ is substituted with at least one substituent selected from the group consisting of OH, OR′, NHR′, NHR′R″, CO₂R′, COR′, CR′═N—OR″, wherein R′ and R″ are each independently H or a C₁₋₆ hydrocarbyl.

Embodiment 5 provides the compound of any one of embodiments 1-4, wherein R¹ is selected from the group consisting of methyl, ethyl, propyl, iso-propyl, butyl, s-butyl, t-butyl, pentyl, hexyl, heptyl, octyl, nonyl, decyl, undecyl, and dodecyl.

Embodiment 6 provides the compound of any one of embodiments 1-5, wherein R¹ is methyl, heptyl, or iso-propyl.

Embodiment 7 provides the compound of any one of embodiments 1-6, wherein R¹ is C₁₋₁₂ heteroalkyl.

Embodiment 8 provides the compound of any one of embodiments 1-7, wherein R¹ is —CH₂—[O—CH₂CH₂]ₚ—OCH₃, wherein p is an integer from 0 to 10.

Embodiment 9 provides the compound of any one of embodiments 1-8, wherein p is 0 or 1.

Embodiment 10 provides the compound of any one of embodiments 1-9, wherein R¹ is C₆₋₁₀ aryl.

Embodiment 11 provides the compound of any one of embodiments 1-10, wherein R¹ has the structure

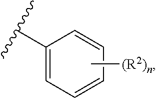

wherein:

n is an integer from 0 to 5,

each occurrence of R² is independently selected from the group consisting of H, F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, C(═NH)N(R)₂, CR═N—N(R)R³, and CR═N—OR³,

each occurrence of R is independently hydrogen, C₁-C₆ alkyl, or C₃-C₈ cycloalkyl, and

R³ is selected from H, [(CH₂)_{q}—O]ᵣ—CH₃, triose, tetrose, pentose, hexose, heptose, and C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, and

q and r are independently integers from 1 to 10.

Embodiment 12 provides the compound of any one of embodiments 1-11, wherein R² is selected from the group consisting of C(═O)CH₃, OH, OCH₃, F, Cl, and Br.

Embodiment 13 provides the compound of any one of embodiments 1-12, wherein R¹ has the structure

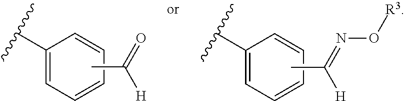

Embodiment 14 provides the compound of any one of embodiments 1-13, wherein R³ is glucose.

Embodiment 15 provides the compound of any one of embodiments 1-14, wherein: [symbol] is a covalent single bond;

R¹ has the structure

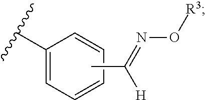

and

Q³ is covalently bonded to Q¹ at the R³ position to form a ring.

Embodiment 16 provides the compound of any one of embodiments 1-15, wherein the compound has an aqueous solubility of greater than 3 μg/mL at about pH 7.

Embodiment 17 provides the compound of any one of embodiments 1-16, wherein the compound is selected from the group consisting of:

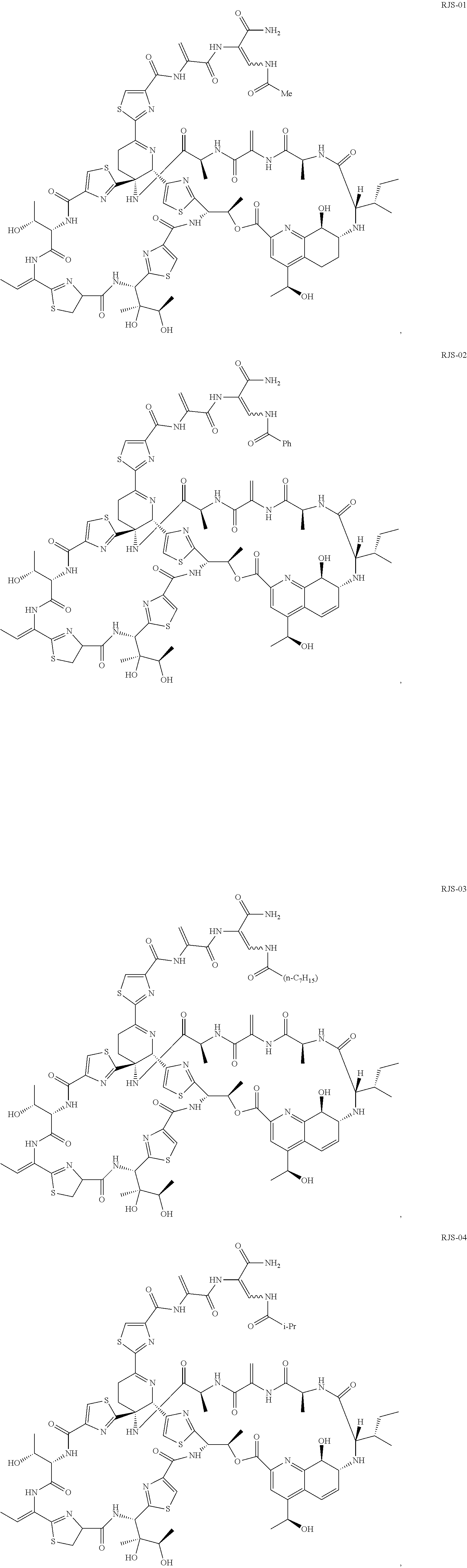

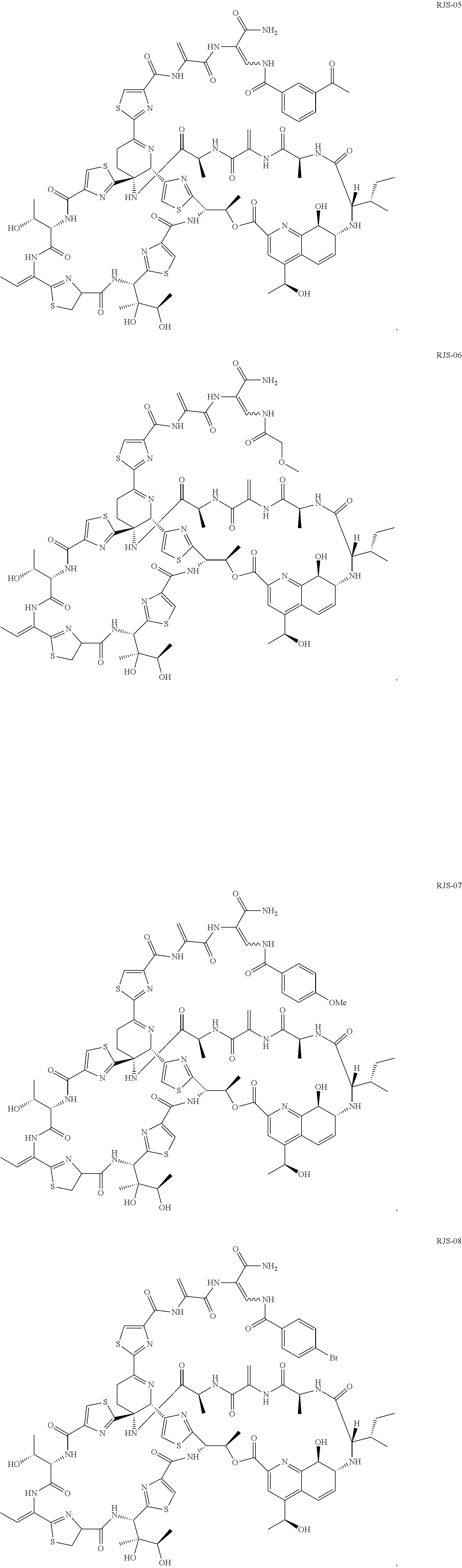

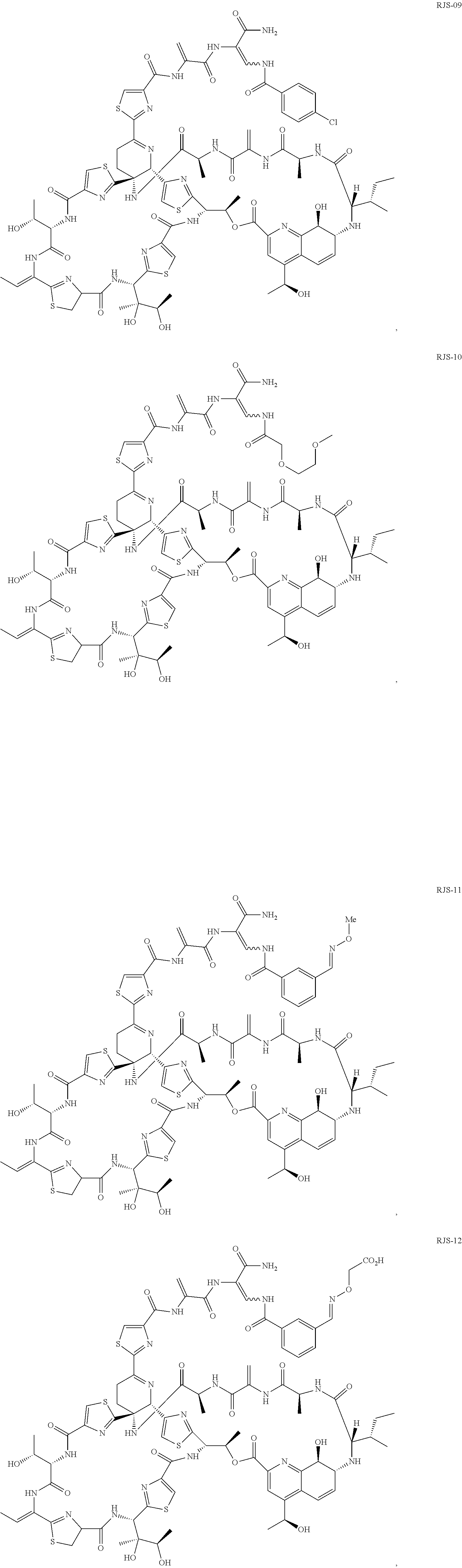

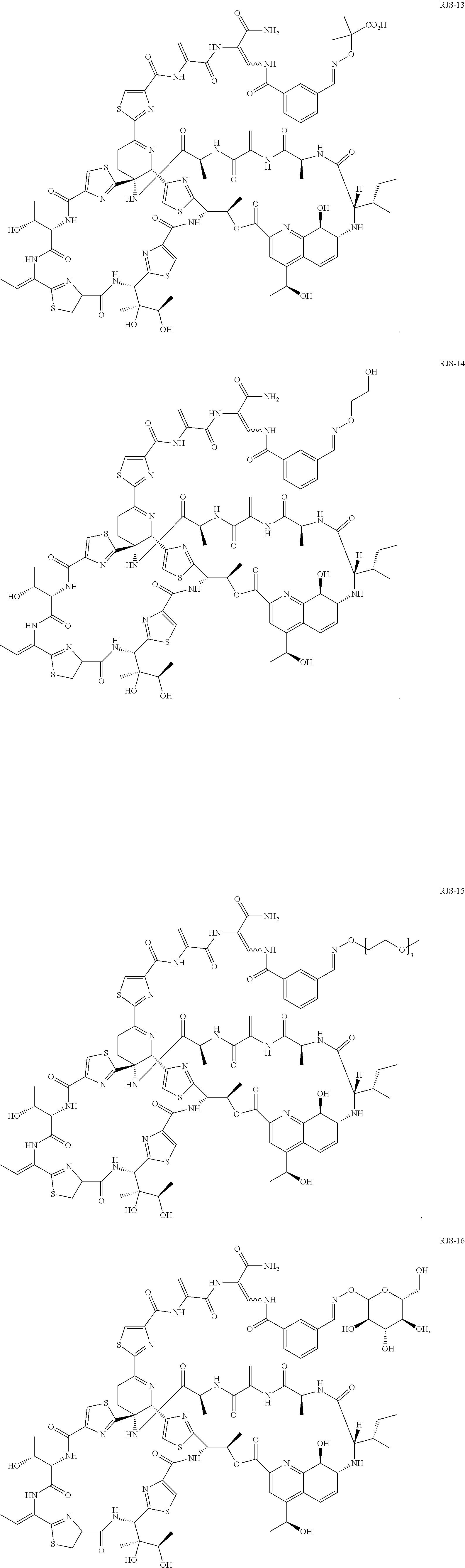

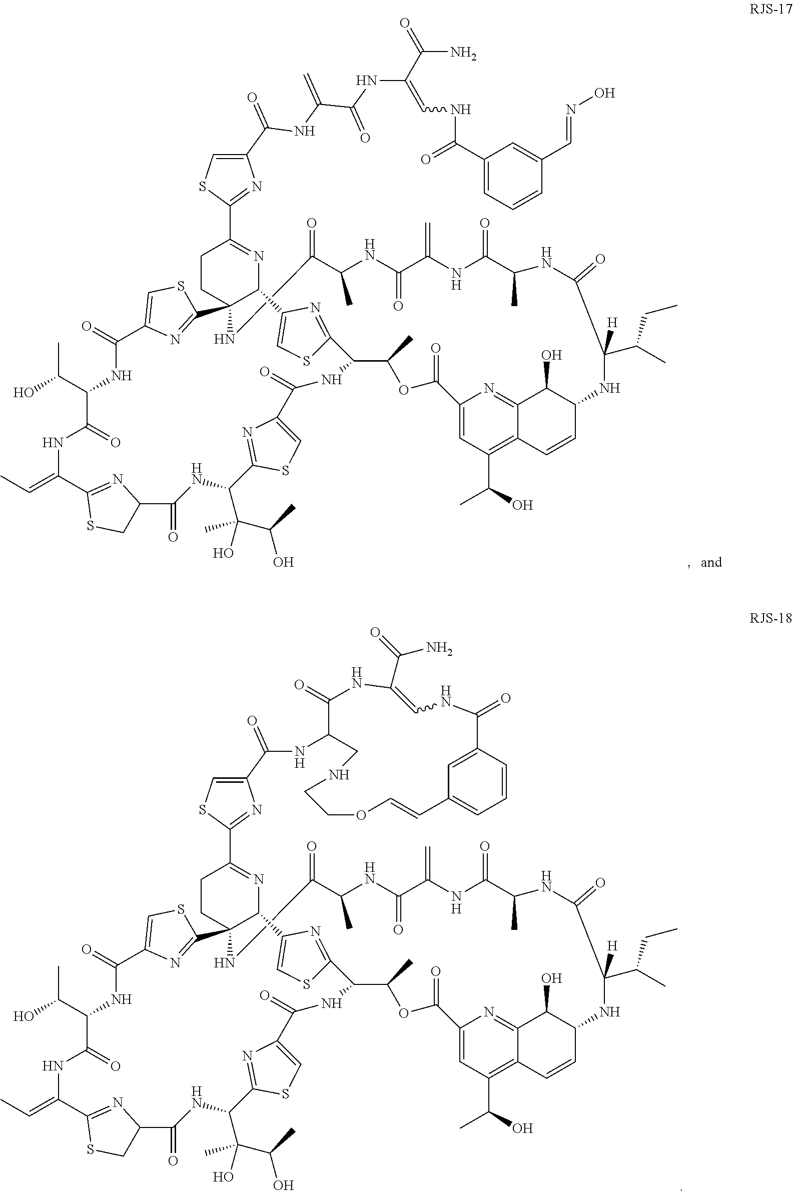

Embodiment 18 provides a method of killing, preventing growth, or disinfecting bacteria, the method comprising contacting the bacteria with a compound of any of embodiments 1-17.

Embodiment 19 provides a method of treating, ameliorating, or preventing a bacterial infection in a subject, the method comprising administering to the subject a therapeutically effective amount of a compound of any of embodiments 1-17.

Embodiment 20 provides the method of any one of embodiments 18-19, wherein at least about 95%, 98%, 99%, 99.1%, 99.2%, 99.3%, 99.4%, 99.5%, 99.6%, 99.7%, 99.8%, 99.9%, 99.99%, 99.999%, or 99.9999% of the bacteria that come in contact with or are exposed to the compound are killed and/or disinfected, and/or their growth prevented.

Embodiment 21 provides the method of any one of embodiments 18-20, wherein the bacteria are pathogenic in mammals.

Embodiment 22 provides the method of any one of embodiments 18-21, wherein the bacteria comprise Gram-positive bacteria.

Embodiment 23 provides the method of any one of embodiments 18-22, wherein the bacteria comprise at least one bacterial genus selected from the group consisting of Bacillus, Clostridium, Corynebacterium, Enterococcus, Listeria, Mycobacterium, Mycoplasma, Staphylococcus, Streptococcus, and Ureaplasma.

Embodiment 24 provides the method of any one of embodiments 18-23, wherein the bacteria comprise methicillin-resistant Staphylococcus aureus (MRSA), vancomycin-resistant Enterococcus faecalis (VRE), vancomycin-sensitive Enterococcus faecalis (VSE), penicillin-resistant S. pneumoniae (PISP), or combinations thereof.

Embodiment 25 provides the method of any one of embodiments 18-24, wherein the compound has an MIC of about 0.01 μg/mL to about 5 μg/mL against the bacteria.

Embodiment 26 provides the method of any one of embodiments 18-25, wherein the subject is a mammal.

Embodiment 27 provides the method of any one of embodiments 18-26, wherein the mammal is human.

Embodiment 28 provides a method of derivatizing a compound comprising a dehydroamino acid residue, the method comprising reacting the compound with R¹

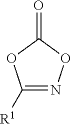

in a solvent system in the presence of a transition metal catalyst of formula [Cp*M(solv)_{d}][SbF₆]ₑ, wherein:

Cp* is a cyclopentadienyl (C₅H₅⁻) anion optionally substituted by 1 to 5 organic groups,

M is a transition metal,

solv is an aprotic solvent,

‘d’ and ‘e’ are independently integers from 1 to 3, wherein 2≤‘d’+‘e’≤5, and

R¹ is H, C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents.

Embodiment 29 provides the method of embodiment 28, wherein the dehydroamino acid residue is a dehydroalanine residue.

Embodiment 30 provides the method of any one of embodiments 28-29, wherein the compound is thiostrepton.

Embodiment 31 provides the method of any one of embodiments 28-30, wherein M is cobalt.

Embodiment 32 provides the method of any one of embodiments 28-31, wherein the transition metal catalyst is [Cp*Co(MeCN)₃][SbF₆]₂.

Embodiment 33 provides the method of any one of embodiments 28-32, wherein the catalyst is present in about 50 mol % with respect to the compound.

Embodiment 34 provides the method of any one of embodiments 28-33, wherein the solvent system comprises a non-polar aprotic solvent and a polar protic solvent.

Embodiment 35 provides the method of any one of embodiments 28-34, wherein the R group comprises an aliphatic or aromatic ketone or aldehyde group.

Embodiment 36 provides the method of any one of embodiments 28-35, wherein the derivatized group is further reacted with a hydroxylamine H₂N—OR₃ under conditions whereby the aliphatic or aromatic ketone or aldehyde group is converted to the corresponding oxime, wherein R³ is selected from the group consisting of H, [(CH₂)_{q}—O]ᵣ—CH₃, triose, tetrose, pentose, hexose, heptose, and C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, wherein q and r are independently integers from 1 to 10.

Embodiment 37 provides the method of any one of embodiments 35, wherein the derivatized group is further reacted with a hydrazine H₂N—N(R)R₃ under conditions whereby the aliphatic or aromatic ketone or aldehyde group is converted to the corresponding hydrazone, wherein R is hydrogen, C₁-C₆ alkyl, or C₃-C₈ cycloalkyl, wherein R³ is selected from the group consisting of H, [(CH₂)_{q}—O]ᵣ—CH₃, triose, tetrose, pentose, hexose, heptose, and C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, and wherein q and r are independently integers from 1 to 10.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of Formula I, or a salt, solvate, enantiomer, tautomer, or diastereomer thereof: 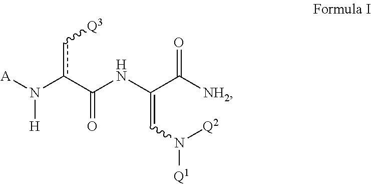
wherein A has the structure: 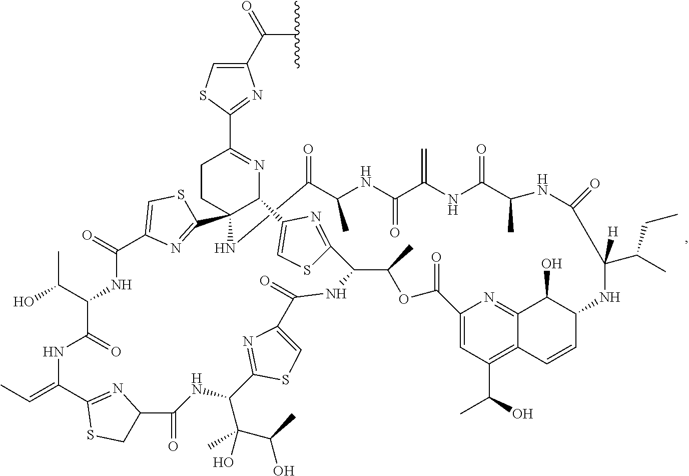
wherein:
Q¹ is H or (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents;
Q² is H; [symbol]
is a covalent single bond or a covalent double bond, wherein
a) if [symbol] is a covalent double bond then Q³ is H or (C═O)—R¹, wherein R¹ is C₁₋₁₂ hydrocarbyl, C₁₋₁₂ heteroalkyl, C₆₋₁₀ aryl, C₃₋₁₀ cycloalkyl, C₃₋₁₀ heterocycloalkyl, or C₆₋₁₀ heteroaryl, each of which is optionally substituted by one or more substituents, provided that if Q³ is H then Q¹ is not H, and if Q³ is not H then Q¹ is H, and
b) if [symbol] is a covalent single bond then Q³ is NH—(CH₂)ₙ—*, n is an integer from 1 to 10, and the * represents a covalent point of attachment to an open valence on Q¹ to form a ring.

2. (canceled)

3. The compound of claim 1, wherein R¹ is C₁₋₁₂ hydrocarbyl.

4. The compound of claim 1, wherein at least one applies:
(a) Q¹ is (C═O)—R¹;
(b) R¹ is substituted with at least one substituent selected from the group consisting of OH, OR′, NHR′, NHR′R″, CO₂R′, COR′, CR′═N—OR″, wherein R′ and R″ are each independently H or a C₁₋₆ hydrocarbyl.

5. The compound of claim 3, wherein R¹ is selected from the group consisting of methyl, ethyl, propyl, iso-propyl, butyl, s-butyl, t-butyl, pentyl, hexyl, heptyl, octyl, nonyl, decyl, undecyl, and dodecyl.

6. The compound of claim 5, wherein R¹ is methyl, heptyl, or iso-propyl.

7. The compound of claim 1, wherein R¹ is C₁₋₁₂ heteroalkyl.

8. The compound of claim 7, wherein R¹ is —CH₂—[O—CH₂CH₂]ₚ—OCH₃, wherein p is an integer from 0 to 10, optionally wherein p is 0 or 1.

9. (canceled)

10. The compound of claim 1, wherein R¹ is C₆₋₁₀ aryl.

11. The compound of claim 10, wherein R¹ has the structure 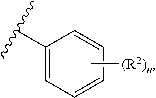
wherein:
n is an integer from 0 to 5,
each occurrence of R² is independently selected from the group consisting of H, F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, C(═NH)N(R)₂, CR═N—N(R)R³, and CR═N—OR³,
each occurrence of R is independently hydrogen, C₁-C₆ alkyl, or C₃-C₈ cycloalkyl, and
R³ is selected from H, [(CH₂)_{q}—O]ᵣ—CH₃, triose, tetrose, pentose, hexose, heptose, and C₁₋₆ hydrocarbyl optionally substituted by 1 to 4 groups independently selected from the group consisting of F, Cl, Br, I, CN, NO, NO₂, ONO₂, azido, CF₃, OCF₃, OR, SR, S(═O)R, S(═O)₂R, R, N(R)₂, OC(═O)N(R)₂, S(═O)₂N(R)₂, SO₃R, C(═O)R, C(═O)OR, OC(═O)R, C(═O)N(R)₂, OC(═O)N(R)₂, (CH₂)₀₋₂N(R)C(═O)R, N(R)S(═O)₂R, N(R)C(═O)OR, N(R)C(═O)R, N(R)C(═O)N(R)₂, and C(═NH)N(R)₂, and
q and r are independently integers from 1 to 10.

12. The compound of claim 11, wherein R² is selected from the group consisting of C(═O)CH₃, OH, OCH₃, F, Cl, and Br.

13. The compound of claim 1, wherein R¹ has the structure

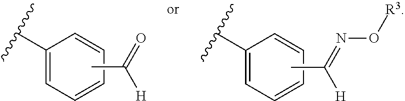

14. The compound of claim 13, wherein R³ is glucose.

15. The compound of claim 1, wherein: [symbol]
is a covalent single bond;
R¹ has the structure 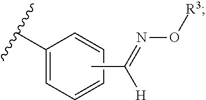
and
Q³ is covalently bonded to Q¹ at the R³ position to form a ring.

16. The compound of claim 1, wherein the compound has an aqueous solubility of greater than 3 μg/mL at about pH 7.

17. The compound of claim 1, wherein the compound is selected from the group consisting of:

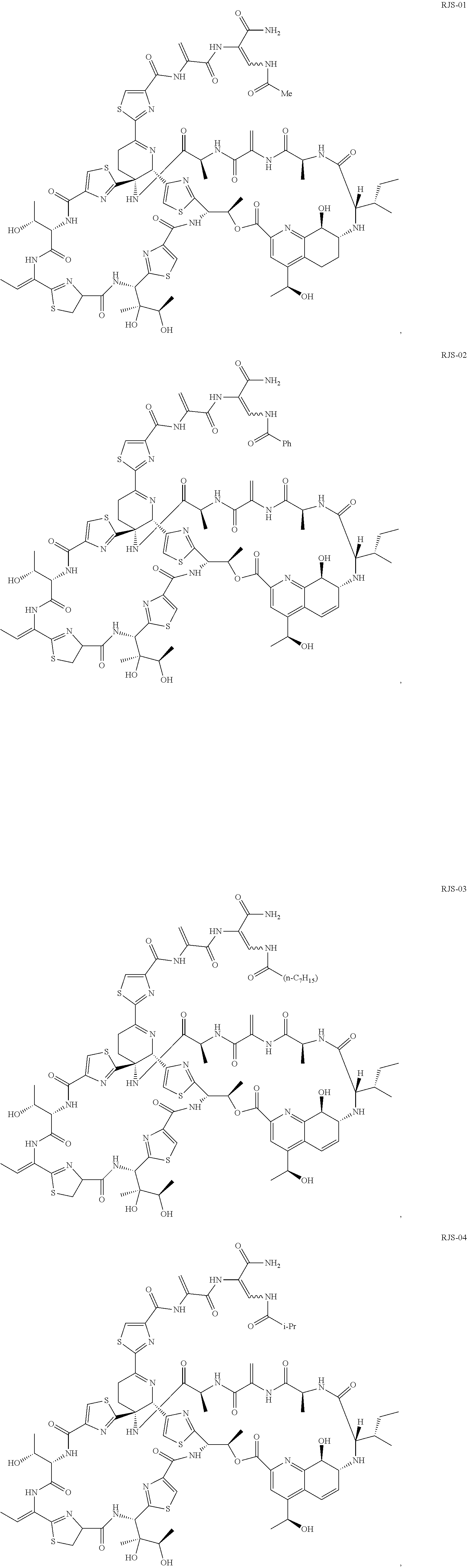

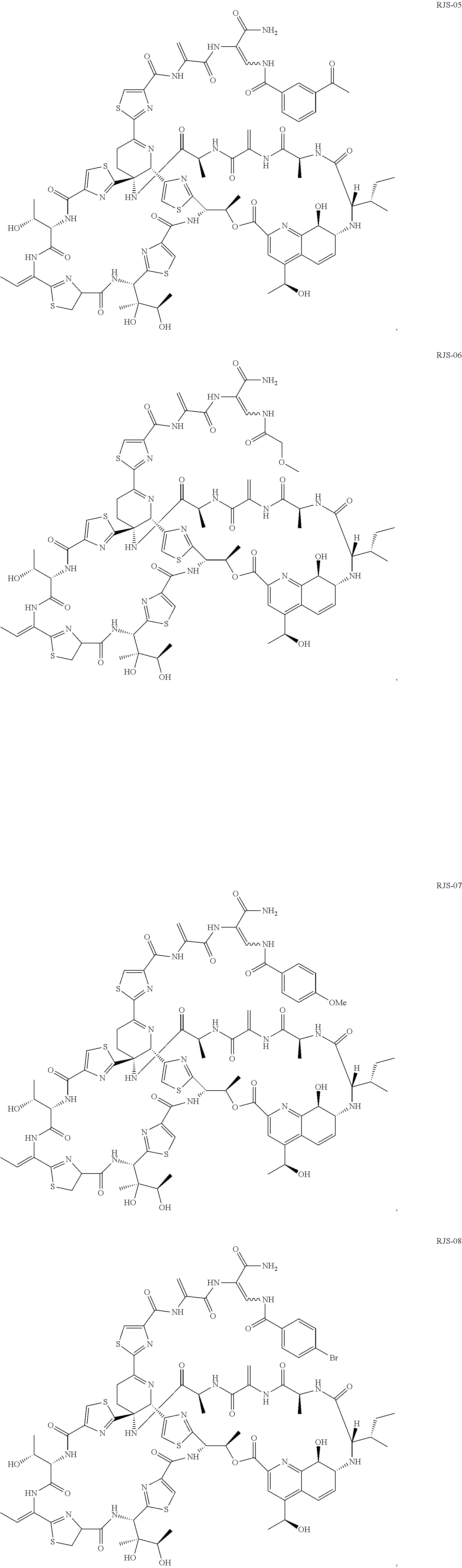

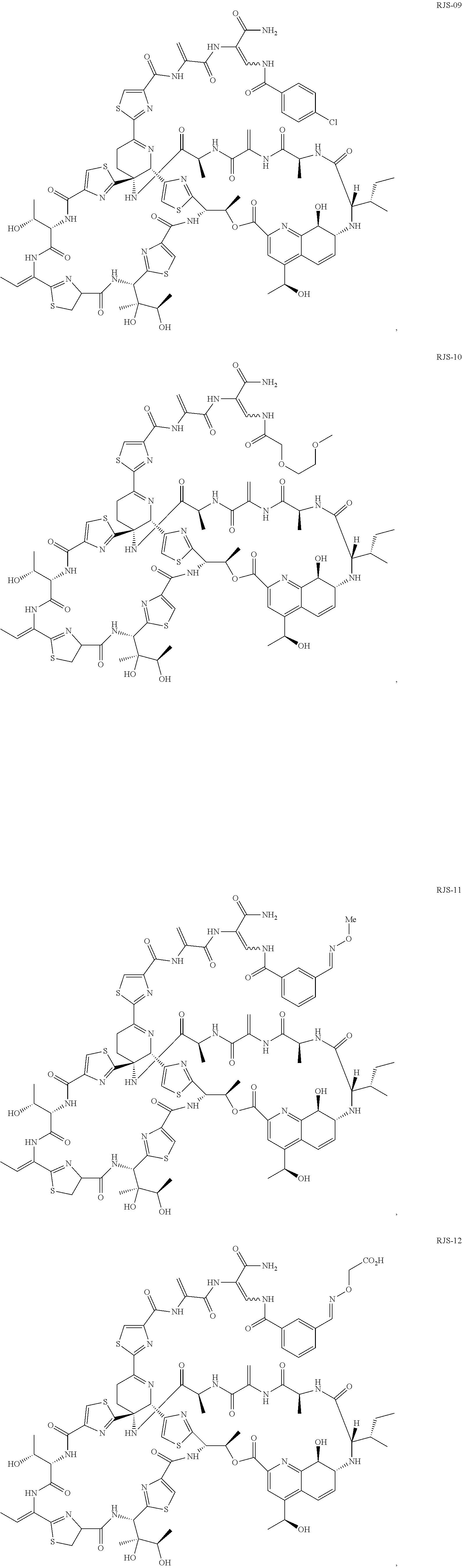

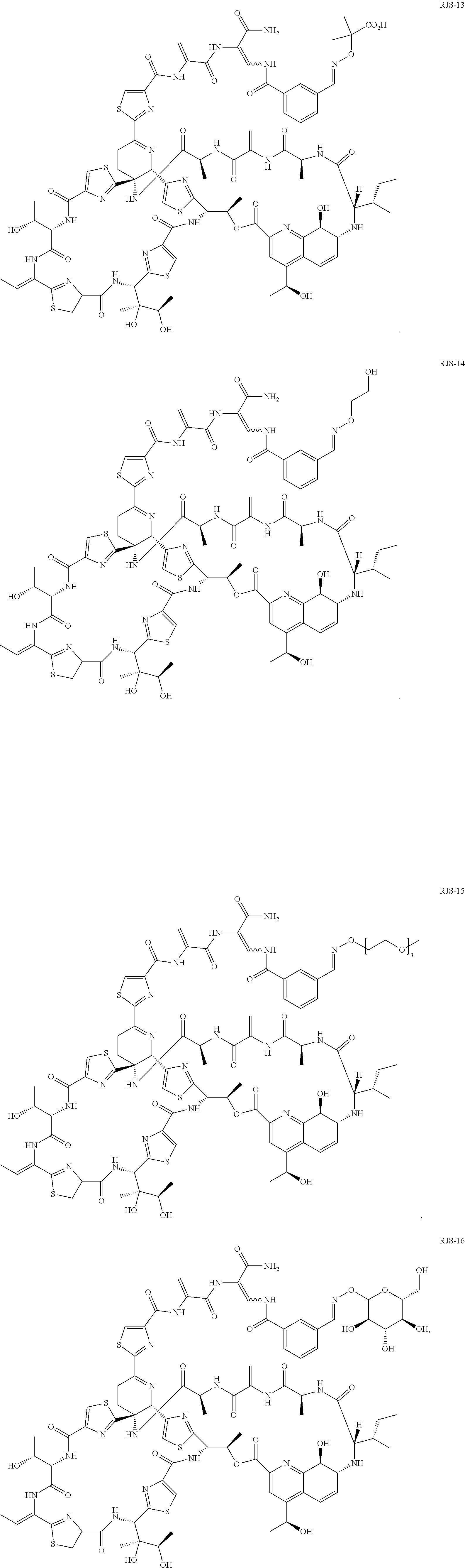

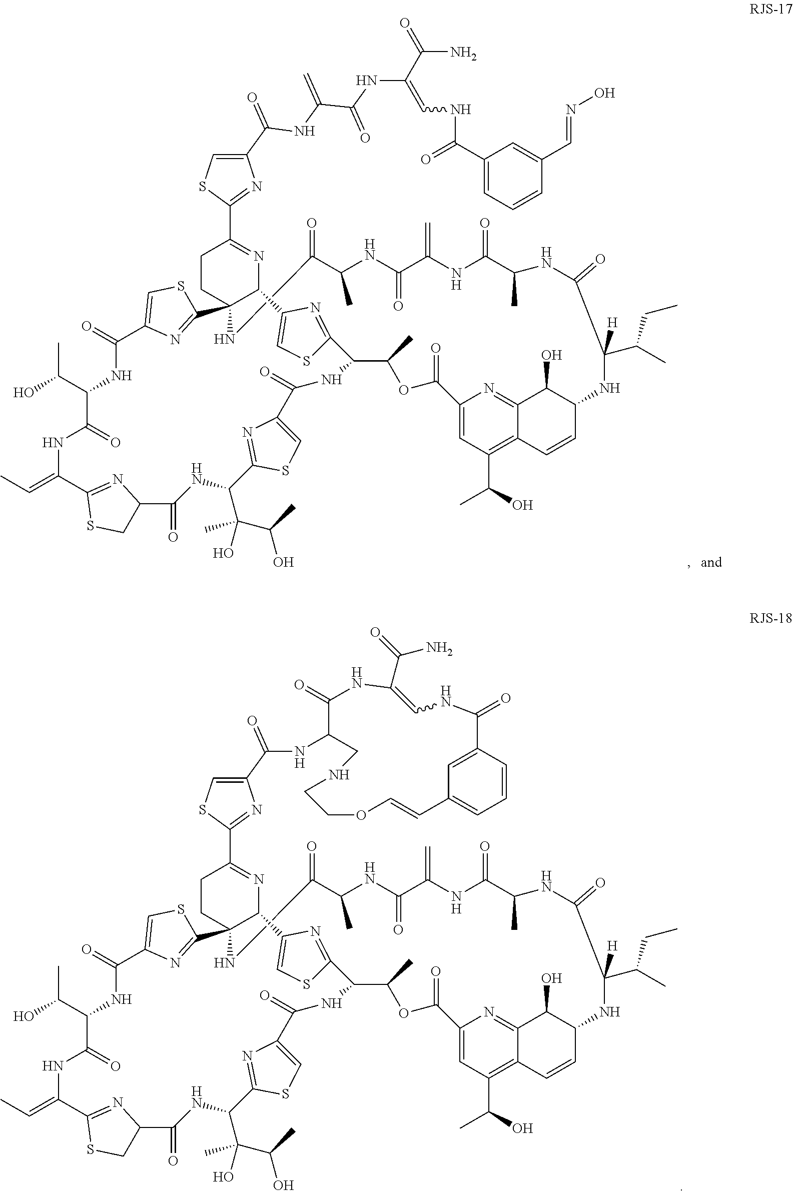

18. A method of killing, preventing growth, or disinfecting bacteria, the method comprising contacting the bacteria with the compound of claim 1.

19. A method of treating or ameliorating a bacterial infection in a subject, the method comprising administering to the subject a therapeutically effective amount of the compound of claim 1.

20. The method of claim 18, wherein at least about 95%, 98%, 99%, 99.1%, 99.2%, 99.3%, 99.4%, 99.5%, 99.6%, 99.7%, 99.8%, 99.9%, 99.99%, 99.999%, or 99.9999% of the bacteria that come in contact with or are exposed to the compound are killed or disinfected.

21. The method of claim 18, wherein the bacteria are at least one of the following:
(a) pathogenic in mammals;
(b) Gram-positive bacteria.

22. (canceled)

23. The method of claim 18, wherein the bacteria comprise at least one bacterial genus selected from the group consisting of Bacillus, Clostridium, Corynebacterium, Enterococcus, Listeria, Mycobacterium, Mycoplasma, Staphylococcus, Streptococcus, and Ureaplasma.

24. The method of claim 18, wherein the bacteria comprise methicillin-resistant Staphylococcus aureus (MRSA), vancomycin-resistant Enterococcus faecalis (VRE), vancomycin-sensitive Enterococcus faecalis (VSE), penicillin-resistant S. pneumoniae (PISP), or combinations thereof.

25. The method of claim 18, wherein the compound has an MIC of about 0.01 μg/mL to about 5 μg/mL against the bacteria.

26.-37. (canceled)
